(19) United States
(12) Patent Application Publication
(10) Pub. No.: US 2016/0235724 A1
(43) Pub. Date: Aug. 18, 2016

(54) TETRAHYDROPYRIDOETHERS FOR THE TREATMENT OF STARGARDT'S DISEASE

(71) Applicant: KATAIRO GMBH, Kusterdingen (DE)

(72) Inventor: ULRICH SCHRAERMEYER, HECHINGEN (DE)

(73) Assignee: KATAIRO GMBH, KUSTERDINGEN (DE)

(21) Appl. No.: 15/027,433

(22) PCT Filed: Oct. 8, 2014
(86) PCT No.: PCT/EP2014/071574

§ 371 Date: Apr. 6, 2016

(30) Foreign Application Priority Data
Oct. 8, 2013 (EP) 13187789.6

(51) Int. Cl. A61K 31/4375

(52) U.S. Cl. CPC A61K 31/4375

(57) ABSTRACT

A pharmaceutical composition comprising tetrahydropyridoethers for use in the treatment of Stargardt's disease.

### CROSS REFERENCE TO PRIOR APPLICATIONS

This application is a U.S. National Phase application under 35 U.S.C. §371 of International Application No. PCT/EP2014/071574, filed on Oct. 8, 2014 and which claims benefit to European Patent Application No. 13187789.6, filed on Oct. 8, 2013. The International application was published in English on Apr. 16, 2015 as WO 2015/052256 A1 under PCT Article 21(2).

### FIELD

The invention relates to tetrahydropyridoethers for the treatment of Stargardt's disease.

### BACKGROUND

### Stargardt's Disease

Stargardt's disease (disease code H35.5 according to ICD-10) is a severe inherited juvenile macular degeneration due to autosomal recessive mutation of the ABCA4 gene. It begins in late childhood. Along with progression of the disease, lipid rich deposits accumulate in the retinal pigment epithelium (RPE) layer beneath the macula. In advanced Stargardt's disease, the buildup of lipofuscin causes atrophy of the RPE and subsequently the macula supplied by this area of the RPE. At the final stage, Stargardt's disease leads to legal blindness.

Stargardt's disease is a rare disease and a recognised distinct medical entity (Blacharski 1988)(Hamel 2007).

Stargardt's disease is symptomatically similar to age-related macular degeneration. On examination, the ophthalmological findings vary significantly with the progression of the disease. In fundus photos, patients with early Stargardt's disease appear to have simple macular degeneration.

The disease is often misdiagnosed, or not diagnosed in the first few years of onset, and this could be the result of little evidence being found during standard eye examinations.

The discovery of the Stargardt gene could help in a test for the direct diagnosis of the disease.

Children with the disease typically begin experiencing visual problems between six and twelve years of age. Symptoms may also first appear in adulthood but are usually diagnosed under the age of twenty. Among those symptoms are reduced vision in dim light, slowed dark adaption, light sensitivity, progressive loss of central vision, yellowish flecks around the macula, macular atrophy and progressive macular dystrophy.

In the early stages, patients may have good visual acuity, but they may experience problems with dark adaption or reading in dim light. Other common symptoms of Stargardt's disease include blurriness and distortion.

At present, there is no approved treatment for patients with Stargardt's disease. Recommendations given by the treating physicians include avoiding UV light and bright blue light, the consumption of antioxidant nutritional supplements such as Vitamin C, zinc, lutein or zeaxanthin or a diet rich of such substances. However, none of these recommendations is capable to stop or drastically delay the progression of the disease (Openshaw, Branham, and Heckenlively 2008; Genead et al. 2009).

### Lipofuscin

The term lipofuscin relates to a lipid rich substance which is found to be accumulated in post mitotic cells of the brain, the heart, or the retinal pigment epithelium in the eye over a life time. The composition is complex and still under investigation. Among the characteristics usually observed are autofluorescence, high content of lipids and a greyish granular appearance when observed in electron microscopic sections. Also, the origin of the substance is unclear in most cases. Therefore, although the substance is always termed as lipofuscin, origin, properties and composition are varying considerably.

### Lipofuscin as Substance “Naturally” Accumulated in Rpe Cells of Healthy Persons

Lipofuscin is a pigment that is formed in tissues with high oxidative stress (heart, liver, brain, eye) (Terman and Brunk 1998) Lipofuscin, also called age pigment, is a brown-yellow, electron-dense, autofluorescent material that accumulates progressively over time in lysosomes of postmitotic cells, such as neurons and cardiac myocytes and the RPE. The exact mechanisms behind this accumulation are still unclear. It can be detected histologically by its autofluorescence properties. The origin of lipofuscin in the RPE is still under debate (Kennedy, Rakoczy, and Constable 1995). Numerous studies indicate that the formation of “natural” lipofuscin is due to the oxidative alteration of macromolecules by oxygen-derived free radicals generated in reactions catalyzed by redox-active iron of low molecular weight. Two principal explanations for the increase of lipofuscin with age have been suggested. The first one is based on the notion that lipofuscin is not totally eliminated (either by degradation or exocytosis) even at a young age, and, thus, accumulates in postmitotic cells as a function of time. Since oxidative reactions are obligatory for life, they would act as age-independent enhancers of lipofuscin accumulation, as well as of many other manifestations of senescence. The second explanation is that the increase of lipofuscin is an effect of aging, caused by an age-related enhancement of autophagocytosis, a decline in intralysosomal degradation, and/or a decrease in exocytosis. No reports state that lipofuscin can be degraded or exocytosed by RPE cells. In the eye, “natural” lipofuscin accumulates with age, especially in the RPE, and occupies a considerable part of the cell volume in elderly persons. Lipofuscin content, expressed as fluorescence intensity, in the macular retinal pigment epithelium (RPE) and choroid was two to three times higher than in other areas, and increased with aging.

### Removal of “Natural” Lipofuscin

For the specific composition of the natural lipofuscin it was known that tetrahydropyridoethers especially, Soraprazan (INN Name) (7R,8R,9R)-2,3-Dimethyl-8-hydroxy-7-(2-methoxyethoxy)-9-phenyl-7,8,9,10-tetrahydro-imidazo [1,2-h][1,7]naphthyridine and its salts and related compounds remove such lipofuscin from RPE cells and can therefore serve as active ingredient in the treatment of AMD degeneration, especially of dry AMD (Julien & Schraermeyer 2012, EP 2080513 A1).

### Stargardt's Related Substance Accumulated in Rpe Cells of Stargardt's Disease Patients

Stargardt's disease is almost always inherited as an autosomal recessive disorder, with only ten percent of cases resulting from a dominant mode of inheritance. Autosomal recessive means that both parents are carriers, having one gene for the disease paired with one normal gene. As a consequence, each of their children has a 25 percent chance of inheriting the two copies of the Stargardt gene (one from each parent) needed to cause the disease. Carriers are unaffected because they have only one copy. At this time, it is impossible to determine who is a carrier for Stargardt's disease until after an affected child is diagnosed. (Burke and Tsang 2011; Vasireddy, Wong, and Ayyagaria 2010)

In 1997, researchers isolated the gene for Stargardt's disease. The ABCA4 gene produces a protein involved in energy transport to and from photoreceptor cells in the retina. Mutations in the ABCA4 gene, which cause Stargardt's disease, produce a dysfunctional protein that cannot perform its transport function. As a result, photoreceptor cells degenerate, and vision loss occurs. One of nineteen mutations in the gene (causing deletions and substitutions of amino acids) has been identified to cause Stargardt's disease. The non-functional ABCA4 protein permits the accumulation of yellow fatty material to accumulate in the retina in young persons. This material causes flecks and, ultimately, loss of vision. Further research is needed to find out how the mutated ABCA4 genes affect the biochemistry of the retina and lead to vision loss (Allikmets et al. 1997; Burke and Tsang 2011).

As the disease progresses, lipid rich deposits accumulate in the retinal pigment epithelium (RPE) layer beneath the macula. They appear as yellowish-tinted flecks. In fluorescence ophthalmoscopy, a substance appears as yellow orange granules accumulating in cells of the RPE. This substance has in some cases also been termed lipofuscin even though its origin and composition is obviously different from that of “natural” lipofuscin observed in aged persons without the gene defect. The RPE is a layer of cells that lies between the retina and the choroid, where it serves the purpose of exchange of nutrients and waste products between the two tissues. RPE cells also account for the recycling of outer segments of the photoreceptors. In advanced Stargardt's disease, the build-up of this type of this substance causes atrophy of the RPE and subsequently the macula supplied by this area of the RPE. The progression of vision loss is variable and can start with a visual acuity of 20/40 and decrease rapidly (especially in children) to 20/200 (legal blindness). By age 50, approximately 50% of patients studied in clinical trials had visual acuities of 20/200 to 20/400. In late stages of this disease, there may also appear colour vision impairment (Chen et al. 2011).

As opposed to natural lipofuscin histological data on the ABCA4−/− mouse model (FIGS. 1 A and B) (Charbel Issa et al. 2013) suggested that in the ABCA4 knock-out mouse yellow fatty material is different from “natural” lipofuscin usually accumulated in healthy monkeys (FIG. 1 C) in human (FIG. 1 D), and in rats (FIG. 1 E).

Transmission electron micrographs of the RPE from an 18-month-old ABCA4^{-/-} mouse are shown at low magnification in FIGS. 1 A and B.

(A) With progression of age, an unusual fuzzy form of lipofuscin with irregular shapes accumulates in the cytoplasm of RPE cells. These lipofuscin-containing organelles are not individual clearly recognizable granules but fuse at many sites (arrows). A melanosome is marked by an arrowhead and Bruch's membrane by an asterisk. The choriocapillaris contains erythrocytes (E). Parts of two nuclei (N) of this RPE cell can be seen on the left and right.

Electron micrographs taken at high magnification (×20000) show the differences between lipofuscin from an 18-month-old ABCA4^{-/-} mouse (FIG. 1 B) and a human eye donor with normal vision (FIG. 1 D) in more detail.

(B) The material accumulated in the cytoplasm of ABCA4^{-/-} mice is irregular in shape and electron density (black arrows). These granules appear to fuse with each other (black arrows) and with melanosomes (white arrows). The electron density of these confluent lipofuscin containing organelles is occasionally as dense as in melanosomes (black arrowhead). A melanosome in a state of disintegration is indicated by a white arrowhead.

(C) Lipofuscin of RPE cells from normal monkeys does not appear as fused granules of lipofuscin and melanolipofuscin. The shape of the granules is more regular than that seen in RPE cells of ABCA4^{-/-} mice (Julien and Schraermeyer 2012).

(D) Lipofuscin in RPE cells from normal human eye donors without Stargardt's disease does not appear as fused granules of lipofuscin and melanolipofuscin. The shape of the granules is more regular than that seen in RPE cells of ABCA4^{-/-} mice.

(E) Lipofuscin of RPE cells from normal rats does not appear as fused granules of lipofuscin and melanolipofuscin. The shape of the granules is more regular than that seen in RPE cells of ABCA4^{-/-} mice.

These results demonstrate that lipofuscin granules of “natural” origin are different in morphology compared to those of the ABCA4^{-/-} mouse model.

The lipofuscin granules of the Stargardt mouse model differ in ultrastructure from those of all normal eyes that accumulate lipofuscin in a “natural” way. They appear in a fuzzy shape and tend to fuse with each other.

The protein composition of lipofuscin was investigated for lipofuscin of different species in the brain (Ottis et al. 2012). The results showed differences in protein composition between species (64% homogeneous proteome) for the same tissue. Proteome analysis was also performed for “natural” lipofuscin from RPE cells ((Ng et al. 2008) and “natural” lipofuscin from brain tissue (Ottis et al. 2012) each of human origin. Differences in lipofuscin composition between different tissues of the same species were more pronounced for this comparison (only 19 out of 49 proteins identical or 38% resemblance) as opposed to the same tissue of different species as stated above.

The chemical composition of lipofuscin granules of humans from “natural” origin was found to differ from the chemical composition of yellow fatty material granules from a Stargardt mouse model.

| | Lipofuscin (at %) | | |
|---|---|---|---|
| | N | O | P |
| Human, | 0.38 ± 0.77 (n = 4) | 4.8 ± 0.14 (n = 6) | 0.25 ± 0.05 (n = 6) |
| 68 years | | | |
| abca4−/− | 3.8 ± 3.3 (n = 3) | 3.2 ± 0.38 (n = 9) | 0.15 ± 0.15 (n = 9) |
| 3 mo | | | |
| t-test | p = 0.1 | p < 0.0001 | p = 0.2 |

Energy-filtered analytical transmission electron microscopy (EDX+EELS; Biesemeier et al., Exp Eye Res. 2011 July; 93(1):29-39.) was used to image the ultrastructure and determine quantitatively the chemical composition of lipofuscin granules of the retinal pigment epithelium of each one human donor (68 years old) and an abca4−/− mouse (3 months old).

Yellow fatty material granules of the abca4−/− Stargardt mouse model showed lower oxygen mole fractions compared to human. Human lipofuscin granules of “natural” origin showed mostly N mole fractions below 1% while most yellow fatty material granules from the Stargardt mouse showed elevated N mole fractions of up to 6%. Also, P mole fractions differed. Here, human lipofuscin granules of “natural” origin were at P mole fractions of 0.25% as opposed to yellow fatty material granules from the Stargardt mouse model with 0.15%.

### SUMMARY

An aspect of the present invention is to provide a compound and pharmaceutical composition for the treatment of Stargardt's disease.

In an embodiment, the present invention provides a pharmaceutical composition comprising tetrahydropyridoethers for use in the treatment of Stargardt's disease. Soraprazan has been found to be effective as an active ingredient in the treatment of Stargardt's disease even though the yellow fatty material is of different origin and different composition.

### BRIEF DESCRIPTION OF THE DRAWINGS

The present invention is described in greater detail below on the basis of embodiments and of the drawings in which:

FIG. 1 A shows Lipofuscin granules and Melanolipofuscin granules of the RPE from a ABCA4−/− mouse eye;

FIG. 1 B shows Lipofuscin granules and Melanolipofuscin granules of the RPE from a ABCA4−/− mouse eye (2000× magnification);

FIG. 1 C shows Lipofuscin granules and Melanolipofuscin granules of the RPE from a Monkey (Makaka fascicularis) eye;

FIG. 1 D shows Lipofuscin granules and Melanolipofuscin granules of the RPE from a Human eye;

FIG. 1 E shows Lipofuscin granules and Melanolipofuscin granules of the RPE from a Rat eye;

FIG. 2 shows under fluorescent light lipofuscin granules (arrows) in RPE of ABCA4−/− mice, which are reduced in number 4 weeks after a single intravitreal injection of 0.32 mg Soraprazan (A) compared to untreated mice (B);

FIG. 3 shows TEM micrographs of RPE cells from ABCA4−/− mice with Soraprazan treatment (A) and untreated (B);

FIG. 4 shows TEM micrographs of the RPE from 12-month-old ABCA4−/− mice after a single intravitreal injection of Soraprazan showing different degree of lipfuscin removal; and

FIG. 5 shows a Quantification of lipofuscin granules by electron microscopy in RPE cells of ABCA4^{-/-} mice.

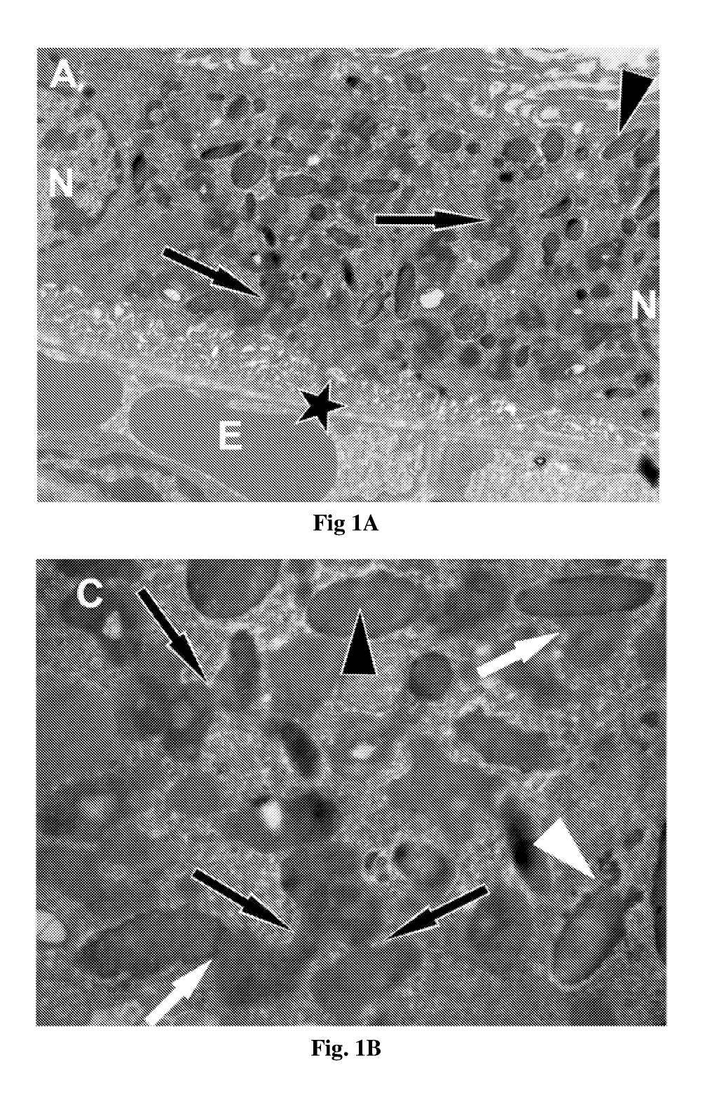

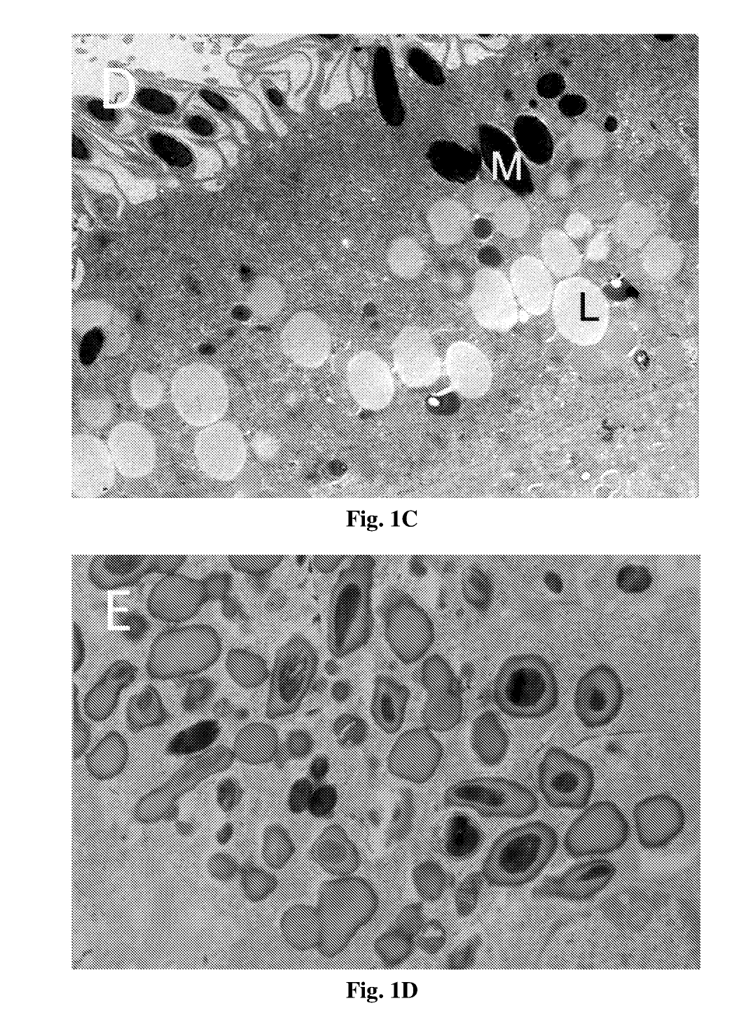

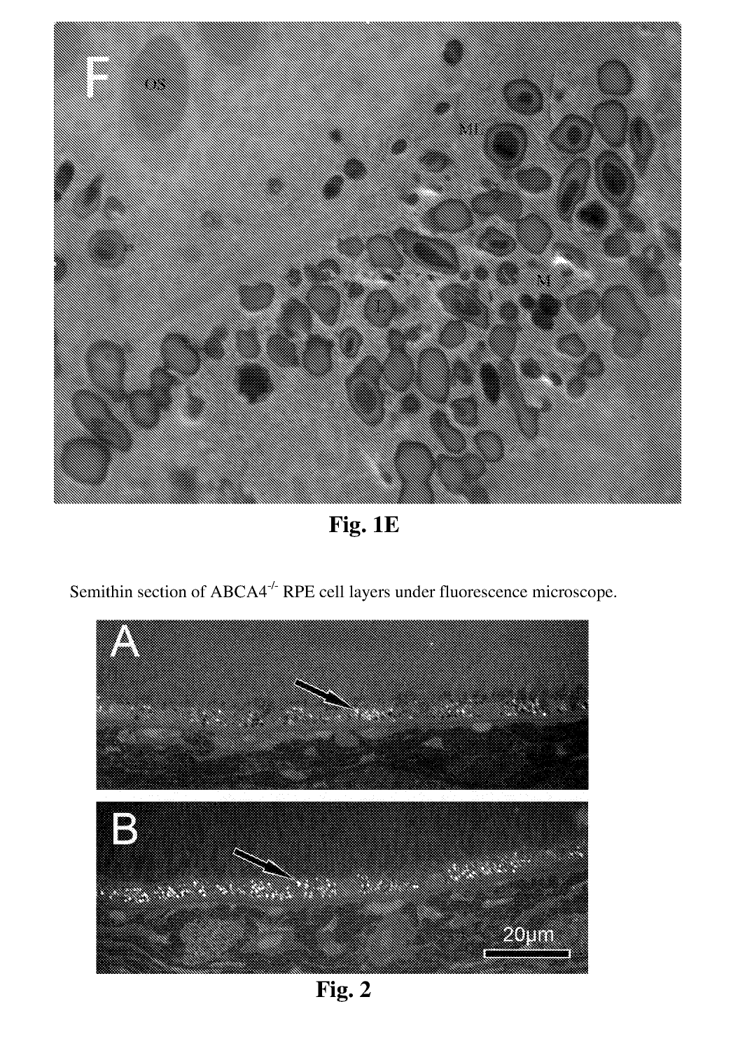

### DETAILED DESCRIPTION

### Compounds

The compounds, including Soraprazan, used for treatment according to the invention have been described in WO 00/17200 (tetrahydropyridoethers) and EP 1 115 725 B1 that are herewith incorporated by reference. EP 1 115 725 B1 especially describes preferred compounds and methods of preparation including starting compounds described e.g. in EP-A-0 299 470 or Kaminski et. al., J. Med. Chem. 1985, 28, 876-892. The compounds according to the invention can be prepared, for example starting from N-protected 8-amino-imidazo[1, 2-a] pyridines in an enantioselective synthesis as described in EP 1 115 725. The full process with different variations and examples is incorporated by reference including the examples of the final products 1-8 and starting compounds A1-D.

The invention furthermore relates to medications, i.e. pharmaceutical compositions, which contain one or more compounds described in EP 1 115 725 and/or their pharmacologically tolerable salts.

These compounds and examples for their preparations are described as follows:

The invention relates to compounds of the formula I

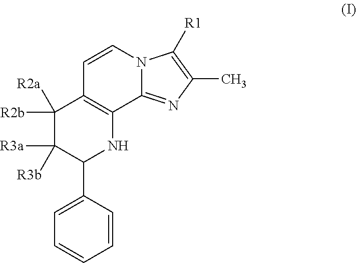

in which

R1 is methyl or hydroxymethyl,

one of the substituents R2a and R2b is hydrogen and the other is hydroxy, methoxy, ethoxy, isopropoxy, methoxyethoxy or methoxypropoxy,

one of the substituents R3a and R3b is hydrogen and the other is hydroxy, methoxy, ethoxy, isopropoxy, methoxyethoxy or methoxypropoxy,

where R2a or R2b on the one hand and R3a or R3b on the other hand are not simultaneously hydroxy, and their salts.

Suitable salts of compounds of the formula I are especially all acid addition salts. Particular mention may be made of the pharmacologically tolerable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in salt preparation—depending on whether a mono- or polybasic acid is concerned and depending on which salt is desired—in an equimolar quantitative ratio or one differing therefrom.

Pharmacologically intolerable salts which can be initially obtained as process products, for example in the preparation of the compounds according to the invention on an industrial scale, are converted into pharmacologically tolerable salts by processes known to the person skilled in the art.

According to expert's knowledge the compounds of the invention as well as their salts may contain, e. g. when isolated in crystalline form, varying amounts of solvents. Included within the scope of the invention are therefore all solvates and in particular all hydrates of the compounds of formula I as well as all solvates and in particular all hydrates of the salts of the compounds of formula I.

The compounds of the formula I have three chiral centers. The invention relates to all eight conceivable stereoisomers in any desired mixing ratio with one another, including the pure enantiomers, which are a preferred subject of the invention.

In a preferred embodiment of the invention compounds are used of the formula 1*

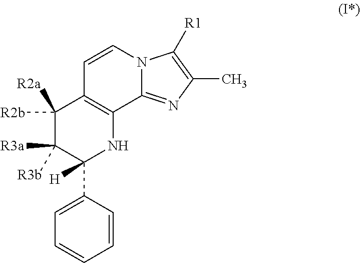

in which

R1 is methyl or hydroxymethyl,

one of the substituents R2a and R2b is hydrogen and the other is hydroxy, methoxy, ethoxy, isopropoxy, methoxyethoxy or methoxypropoxy,

one of the substituents R3a and R3b is hydrogen and the other is hydroxy, methoxy, ethoxy, isopropoxy, methoxyethoxy or methoxypropoxy,

where R2a or R2b on the one hand and R3a or R3b on the other hand are not simultaneously hydroxy,

and their salts.

An embodiment (embodiment a) of the invention are compounds of the formula 1*,

in which

R1 is methyl,

one of the substituents R2a and R2b is hydrogen and the other is methoxy, ethoxy, isopropoxy, methoxyethoxy or methoxypropoxy,

one of the substituents R3a and R3b is hydrogen and the other is hydroxy,

and their salts.

A further embodiment (embodiment b) of the invention are compounds of the formula 1*,

in which

R1 is methyl,

one of the substituents R2a and R2b is hydrogen and the other is hydroxy,

one of the substituents R3a and R3b is hydrogen and the other is methoxy, ethoxy, isopropoxy, methoxyethoxy or methoxypropoxy,

and their salts.

A further embodiment (embodiment c) of the invention are compounds of the formula I*,

in which

R1 is methyl,

one of the substituents R2a and R2b is hydrogen and the other is methoxy, ethoxy, isopropoxy, methoxyethoxy or methoxypropoxy,

one of the substituents R3a and R3b is hydrogen and the other is methoxy, ethoxy, isopropoxy, methoxyethoxy or methoxypropoxy,

and their salts.

A further embodiment (embodiment d) of the invention are compounds of the formula 1*,

in which

R1 is hydroxymethyl,

one of the substituents R2a and R2b is hydrogen and the other is methoxy, ethoxy, isopropoxy, methoxyethoxy or methoxypropoxy,

one of the substituents R3a and R3b is hydrogen and the other is hydroxy,

and their salts.

A further embodiment (embodiment e) of the invention are compounds of the formula 1*,

in which

R1 is hydroxymethyl,

one of the substituents R2a and R2b is hydrogen and the other is hydroxy,

one of the substituents R3a and R3b is hydrogen and the other is methoxy, ethoxy, isopropoxy, methoxyethoxy or methoxypropoxy,

and their salts.

A further embodiment (embodiment f) of the invention are compounds of the formula 1*,

in which

R1 is hydroxymethyl,

one of the substituents R2a and R2b is hydrogen and the other is methoxy, ethoxy, isopropoxy, methoxyethoxy or methoxypropoxy,

one of the substituents R3a and R3b is hydrogen and the other is methoxy, ethoxy, isopropoxy, methoxyethoxy or methoxypropoxy,

and their salts.

Preferred compounds of the embodiments a to f are those, in which R3b is hydrogen.

Particularly preferred compounds of the embodiments a to f are those, in which R2a and R3b are hydrogen.

Preferred compounds within the scope of the invention are those of embodiment a, which can be characterized by the formula 1**

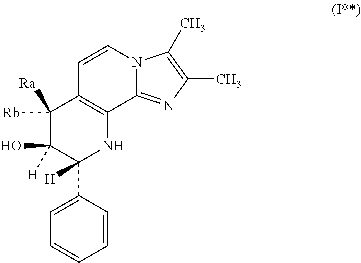

in which

one of the substituents Ra and Rb is hydrogen and the other is methoxy, ethoxy, isopropoxy, methoxyethoxy or methoxypropoxy,

and their salts.

Particularly preferred compounds of embodiment a are those of formula 1**, in which

Ra is hydrogen and

Rb is methoxy, ethoxy, isopropoxy, methoxyethoxy or methoxypropoxy,

and their salts.

With the aid of the general formula I*, the following exemplary preferred compounds according to the invention may actually be mentioned by means of the substituent meanings for R1, R2a, R2b, R3a and R3b in the following Table 1:

TABLE 1

| R1 | R2a | R2b | R3a | R3b |
|---|---|---|---|---|
| CH₃ | H | OCH₃ | OH | H |
| CH₃ | H | OC₂H₅ | OH | H |
| CH₃ | H | OCH(CH₃)₂ | OH | H |
| CH₃ | H | OCH₂CH₂OCH₃ | OH | H |
| CH₃ | H | OCH₂CH₂CH₂OCH₃ | OH | H |
| CH₃ | H | OH | OCH₃ | H |
| CH₃ | H | OH | OC₂H₅ | H |
| CH₃ | H | OH | OCH(CH₃)₂ | H |
| CH₃ | H | OH | OCH₂CH₂OCH₃ | H |
| CH₃ | H | OH | OCH₂CH₂CH₂OCH₃ | H |
| CH₃ | H | OCH₃ | OCH₃ | H |
| CH₃ | H | OC₂H₅ | OC₂H₅ | H |
| CH₃ | H | OCH(CH₃)₂ | OCH(CH₃)₂ | H |
| CH₃ | H | OCH₂CH₂OCH₃ | OCH₂CH₂OCH₃ | H |
| CH₃ | H | OCH₂CH₂CH₂OCH₃ | OCH₂CH₂CH₂OCH₃ | H |
| CH₂OH | H | OCH₃ | OH | H |
| CH₂OH | H | OC₂H₅ | OH | H |
| CH₂OH | H | OCH(CH₃)₂ | OH | H |
| CH₂OH | H | OCH₂CH₂OCH₃ | OH | H |
| CH₂OH | H | OCH₂CH₂CH₂OCH₃ | OH | H |
| CH₂OH | H | OH | OCH₃ | H |
| CH₂OH | H | OH | OC₂H₅ | H |
| CH₂OH | H | OH | OCH(CH₃)₂ | H |
| CH₂OH | H | OH | OCH₂CH₂OCH₃ | H |
| CH₂OH | H | OH | OCH₂CH₂CH₂OCH₃ | H |
| CH₂OH | H | OCH₃ | OCH₃ | H |
| CH₂OH | H | OC₂H₅ | OC₂H₅ | H |
| CH₂OH | H | OCH(CH₃)₂ | OCH(CH₃)₂ | H |
| CH₂OH | H | OCH₂CH₂OCH₃ | OCH₂CH₂OCH₃ | H |
| CH₂OH | H | OCH₂CH₂CH₂OCH₃ | OCH₂CH₂CH₂OCH₃ | H |
| CH₃ | OCH₃ | H | OH | H |
| CH₃ | OC₂H₅ | H | OH | H |
| CH₃ | OCH(CH₃)₂ | H | OH | H |
| CH₃ | OCH₂CH₂OCH₃ | H | OH | H |
| CH₃ | OCH₂CH₂CH₂OCH₃ | H | OH | H |
| CH₃ | OH | H | OCH₃ | H |
| CH₃ | OH | H | OC₂H₅ | H |
| CH₃ | OH | H | OCH(CH₃)₂ | H |
| CH₃ | OH | H | OCH₂CH₂OCH₃ | H |
| CH₃ | OH | H | OCH₂CH₂CH₂OCH₃ | H |
| CH₃ | OCH₃ | H | OCH₃ | H |
| CH₃ | OC₂H₅ | H | OC₂H₅ | H |
| CH₃ | OCH(CH₃)₂ | H | OCH(CH₃)₂ | H |
| CH₃ | OCH₂CH₂OCH₃ | H | OCH₂CH₂OCH₃ | H |
| CH₃ | OCH₂CH₂CH₂OCH₃ | H | OCH₂CH₂CH₂OCH₃ | H |
| CH₂OH | OCH₃ | H | OH | H |
| CH₂OH | OC₂H₅ | H | OH | H |
| CH₂OH | OCH(CH₃)₂ | H | OH | H |
| CH₂OH | OCH₂CH₂OCH₃ | H | OH | H |
| CH₂OH | OCH₂CH₂CH₂OCH₃ | H | OH | H |
| CH₂OH | OH | H | OCH₃ | H |
| CH₂OH | OH | H | OC₂H₅ | H |
| CH₂OH | OH | H | OCH(CH₃)₂ | H |
| CH₂OH | OH | H | OCH₂CH₂OCH₃ | H |
| CH₂OH | OH | H | OCH₂CH₂CH₂OCH₃ | H |
| CH₂OH | OCH₃ | H | OCH₃ | H |
| CH₂OH | OC₂H₅ | H | OC₂H₅ | H |
| CH₂OH | OCH(CH₃)₂ | H | OCH(CH₃)₂ | H |
| CH₂OH | OCH₂CH₂OCH₃ | H | OCH₂CH₂OCH₃ | H |
| CH₂OH | OCH₂CH₂CH₂OCH₃ | H | OCH₂CH₂CH₂OCH₃ | H |

and the salts of these compounds.

The compounds according to the invention can be prepared as described by way of example in the following examples, or using analogous process steps starting from appropriate starting compounds (see, for example, EP-A-0 299 470 or Kaminski et al., J. Med. Chem. 1985, 28, 876-892). The starting compounds are known or can be prepared analogously to the known compounds. The compounds according to the invention can be prepared for example starting from N-protected 8-amino-imidazo[1,2-a]pyridines according to the following reaction scheme:

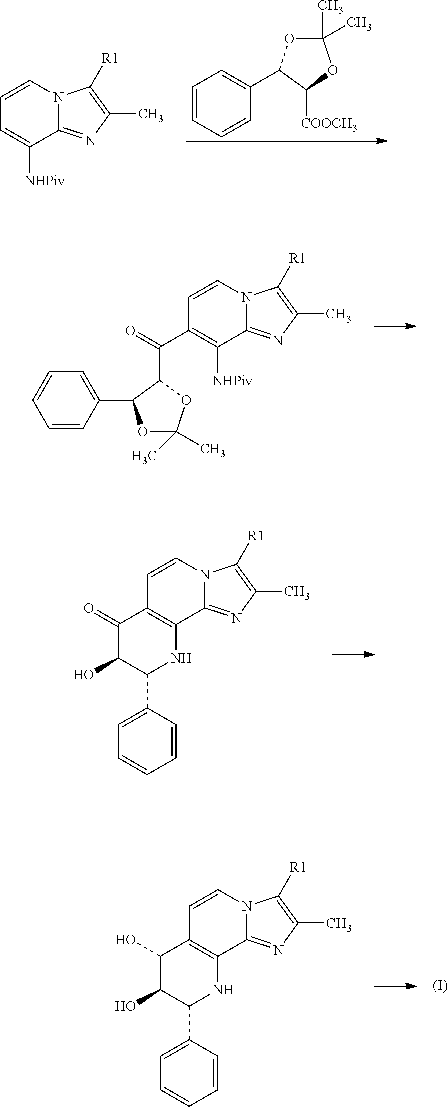

The above scheme represents an example of an enantioselective synthesis. The N-protected (Piv represents a customary protective group, preferably the pivaloyl group), 8-aminoimidazo [1,2-a]pyridine deprotonated in the 7-position is reacted with an enantiomerically pure dioxolane. This initially leads to a condensation product which can be cyclized under strongly acidic conditions with removal of the protecting groups. The subsequent reduction of the keto group using sodium borohydride leads in over 90% enantiomeric purity to the 7,8-trans-diol indicated. The subsequent etherification which is carried out according to known processes, e. g. as described in the Examples, leads to the final products of formula 1* in which R2a and R3b are hydrogen. The corresponding 7,8-cis-compound is obtained from the mother liquor, which is left after separating off the 7,8-trans-compound, by chromatographic purification.

The substances according to the invention are isolated and purified in a manner known per se, for example, by distilling off the solvent in vacuo and recrystallizing the residue obtained from a suitable solvent or subjecting it to one of the customary purification methods, such as, for example, column chromatography on suitable support material.

Salts are obtained by dissolving the free compound in a suitable solvent, e. g. in a chlorinated hydrocarbon, such as dichloromethane or chloroform, or a low molecular weight aliphatic alcohol (ethanol, isopropanol) which contains the desired acid, or to which the desired acid is subsequently added. The salts are obtained by filtering, reprecipitating, precipitating with a nonsolvent for the addition salt or by evaporating the solvent. Salts obtained can be converted by alkalization or by acidification into the free compounds, which in turn can be converted into salts. In this way, pharmacologically intolerable salts can be converted into pharmacologically tolerable salts.

The pure enantiomers, in particular the pure enantiomers of the formula I*, to which the invention preferably relates, can be obtained in a manner familiar to the person skilled in the art, for example by enantioselective synthesis (see, for example, the Scheme), by chromatographic separation on chiral separating columns, by derivatization with chiral auxiliary reagents, subsequent separation of diastereomers and removal of the chiral auxiliary group, by salt formation with chiral acids, subsequent separation of the salts and liberation of the desired compound from the salt, or by (fractional) crystallization from a suitable solvent. Trans-products obtained (with R2a and R3b=hydrogen) can be converted (at least partly) to the corresponding cis-products (with R2b and R3b=hydrogen) by standing under acidic conditions (e. g. 2 equivalents of acid, such as sulfuric acid) in the corresponding alcohol R2a-OH. Likewise, cis-products obtained can be converted to the corresponding trans-products. The cis- and trans-products are separated e. g. by chromatography or by crystallization.

The following examples serve to illustrate the invention further without restricting it. Likewise, further compounds of the formula I whose preparation is not described explicitly can be prepared analogously or in a manner familiar to the person skilled in the art using customary process techniques. The abbreviation min stands for minute(s), h for hour(s) and ee for enantiomeric excess.

The pharmaceutical composition can be prepared and administered by the skilled person based on his common general knowledge in the respective field.

44 different retinal areas of ABCA4 knock-out mice—a disease model for Stargardt's disease—were investigated for their lipofuscin content after intra-vitreal injection with Soraprazan or as control. Lipofuscin content of RPE cells was significantly reduced in treated animals.

The results demonstrate that administration of Soraprazan induces lipofuscin elimination from RPE cells that originated from a mutation of the ABCA4 gene.

### EXAMPLES

### Final Products

### 1A. (7R,8R,9R)-2,3-Dimethyl-8-hydroxy-7-methoxy-9-phenyl-7,8,9,10-tetrahydro-imidazo[1,2-h][1,7] naphthyridine

### Method a

20 g (65 mmol) of (7R,8R,9R)-2,3-dimethyl-7,8-dihydroxy-9-phenyl-7,8,9,10-tetrahydro-imidazo [1,2-h][1,7] naphthyridine are dissolved in methanol (350 ml). 13.5 g of sulfuric acid are added and the solution is stirred for 48 h at 50 C. After cooling the reaction mixture is poured into 250 ml of water. The pH is adjusted by aqueous saturated sodium hydrogen carbonate solution to neutral pH. The precipitate is collected and purified on silica gel (eluent: diethylether). 2.5 g of the title compound are obtained as colourless crystals of melting point 164-165° C. (2-propanol).

### Method b

10 g (32.5 mmol) of (7R,8R,9R)-2,3-dimethyl-7,8-dihydroxy-9-phenyl-7,8,9,10-tetrahydro-imidazo [1,2-h][1,7] naphthyridine are dissolved in 200 ml of dry dimethylformamide. 1.9 g of commercially available sodium hydride in paraffin (80%) are added in small portions at room temperature. After 1 h 9.1 g (65 mmol) of methyl iodide, dissolved in 4 ml of dimethylformamide, are added and the mixture is stirred for an additional hour. The reaction mixture is poured into cold water. 20 ml of a saturated aqueous ammonium chloride solution is added; the yellow precipitate is collected and discarded. The filtrate is extracted several times with ethyl acetate, the combined organic phases are washed several times with water and the solvent is evaporated in vacuo. The solid residue is purified on silica gel (diethylether).

2 g of the title compound are obtained as colourless crystals of melting point 164-165° C. (2-propanol).

### 1B. (7S,8S,9S)-2,3-Dimethyl-8-hydroxy-7-methoxy-9-phenyl-7,8,9,10-tetrahydro-imidazo [1,2-h][1,7] naphthyridine

The title compound of melting point 161-162° C. is obtained similarly to the procedure described in Example 1, Method a, using (7S,8S,9S)-2,3-Dimethyl-7,8-dihydroxy-9-phenyl-7,8,9,10-tetrahydroimidazo [1,2h][1,7] naphthyridine as starting material.

### 2A. (7S,8R,9R)-2,3-Dimethyl-8-hydroxy-7-methoxy-9-phenyl-7,8,9,10-tetrahydro-imidazo [1,2-h][1,7]naphthyridine

6 g of the title compound are obtained as colourless powder of melting point 108-110° C. after purification on silica gel according to Example 1A, Method a, starting from (7S,8R, 9R)-2,3-Dimethyl-7,8-dihydroxy-9-phenyl-7,8,9,10-tetrahydro-imidazo [1,2-h][1,7] naphthyridine.

### 2B. (7R,8S,9S)-2,3-Dimethyl-8-hydroxy-7-methoxy-9-phenyl-7,8,9,10-tetrahydro-imidazo[1,2-h][1,7]naphthyridine

The title compound of melting point 171-172° C. is obtained from the mother liquor of Example 1B after purification on silica gel (eluent: diethyl ether).

### 3. (7R,8R,9R)-2,3-Dimethyl-7-ethoxy-8-hydroxy-9-phenyl-7,8,9,10-tetrahydro-imidazo [1,2-h][1,7]naphthyridine

500 mg of the title compound are obtained by reaction of (7R,8R,9R)-2,3-dimethyl-7,8-dihydroxy-9-phenyl-7,8,9,10-tetrahydro-imidazo[1,2-h][1,7]naphthyridine with ethanol and sulfuric acid according to Example 1, Method a, after purification on silica gel (eluent: diethylether). Melting point: 188-190° C.

### 4. (7S,8R,9R)-2,3-Dimethyl-7-ethoxy-8-hydroxy-9-phenyl-7,8,9,10-tetrahydro-imidazo [1,2-h][1,7]naphthyridine

800 mg of the title compound of melting point 135-137° C. are obtained as a solid by further purification of the mother liquor of Example 3 on silica gel.

### 5A. (7R,8R,9R)-2,3-Dimethyl-8-hydroxy-7-(2-methoxyethoxy)-9-phenyl-7,8,9,10-tetrahydro-imidazo[1,2-h][1,7] naphthyridine

### Method a

5 g of the title compound of melting point 130-131° C. are obtained by reaction of 20 g (7R,8R,9R)-2,3-dimethyl-7,8-dihydroxy-9-phenyl-7,8,9,10-tetrahydro-imidazo [1,2-h][1,7] naphthyridine with 2-methoxy-ethanol according to Example 1, Method a.

### Method b

To a solution of 100 g of (7R,8R,9R)-2,3-Dimethyl-7,8-dihydroxy-9-phenyl-7,8,9,10-tetrahydroimidazo[1,2h][1,7]naphthyridine in 1 L of 2-ethoxyethanol, 64 g of concentrated. sulfuric acid are added slowly at room temperature under an argon atmosphere. The rate of addition is such that the temperature of the mixture does not exceed 35° C. After further 15 hours of stirring at room temperature the greenish solution is poured into a mixture of 1 kg of crushed ice and 800 ml of dichloromethane. The pH of the stirred mixture is adjusted to 7.5 by addition of a 10 M aqueous sodium hydroxide solution, the organic layer is separated off, the aqueous layer is extracted three times with dichloromethane (200 ml each), the dichloromethane layers are washed collectively with 500 ml of water (six times) and are then dried over sodium sulfate. After complete evaporation of the solvent under reduced pressure the remaining oily residue is treated with 450 ml of acetone to yield 75 g off-white crystals consisting of a 1:1 mixture of the title compound and its (7S,8R,9R)-epimer. The mixture is separated by preparative HPLC using methanol as eluent. 28 g of the title compound of melting point 128-129° C. are obtained after recrystallization from ethyl acetate.

### 5B. (7S,8S,9S)-2,3-Dimethyl-8-hydroxy-7-(2-methoxyethoxy)-9-phenyl-7,8,9,10-tetrahydro-imidazo [1,2-h][1,7]naphthyridine

The title compound of melting point 130-131° C. is obtained similarly to the procedure described in Example 5A, Method a, using (7S,8S,9S)-2,3-Dimethyl-7,8-dihydroxy-9-phenyl-7,8,9,10-tetrahydroimidazo[1,2h][1,7]naphthyridine as starting material.

### 6A. (7S,8R,9R)-2,3-Dimethyl-8-hydroxy-7-(2-methoxyethoxy)-9-phenyl-7,8,9,10-tetrahydro-imidazo[1,2][1,7]naphthyridine

7.8 g of the title compound of melting point 131-132° C. are obtained as a solid from the mother liquor of Example 5A after purification on silica gel (eluent: diethyl ether).

### 6B. (7R,8S,9S)-2,3-Dimethyl-8-hydroxy-7-(2-methoxyethoxy)-9-phenyl-7,8,9,10-tetrahydro-imidazo[1,2-h][1,7] naphthyridine

The title compound of melting point 131-132° C. is obtained from the mother liquor of Example 5B after purification on silica gel (eluent: diethyl ether).

### 7. (7S,8R,9R)-2,3-Dimethyl-8-hydroxy-9-phenyl-7-(2-propoxy)-7,8,9,10-tetrahydro-imidazo [1,2-h][1,7]naphthyridine

1 g of the title compound of melting point 168-9 C is obtained by reaction of 3 g of (7R,8R,9R)-2,3-di-methyl-7,8-dihydroxy-9-phenyl-7,8,9,10-tetrahydro-imidazo [1,2-h][1,7] naphthyridine with 2-propanol according to Example 1, Method a.

### 8. (7R,8R,9R)-2,3-Dimethyl-7,8-dimethoxy-9-phenyl-7,8,9,10-tetrahydro-imidazo[1,2-h][1,7]naphthyridine

8 g of the title compound of melting point 155-156° C. are obtained by reaction of 10 g of (7R,8R,9R)-2,3-dimethyl-7,8-dihydroxy-9-phenyl-7,8,9,10-tetrahydro-imidazo[1,2-h][1,7] naphthyridine with 1.9 g of sodium hydride (80%) and 9.1 g of methyl iodide according to Example 1, Method b.

### Starting Compounds

### A1. 2,3-Dimethyl-7-[(2R,3S)-2,3-O-isopropylidene-3-phenylpropan-1-on-1-yl]-8-pivaloylamino-imidazo[1,2-a]pyridine

60 g (0.245 mol) of 2,3-dimethyl-8-pivaloylaminoimidazo[1,2-a]pyridine are dissolved in 1.5 L of anhydrous diethyl ether with exclusion of moisture and under an argon atmosphere and cooled to −75° C. By means of a flex needle, 408 ml (0.612 mol) of tert-butyllithium solution (1.5 M in n-pentane) are added dropwise such that the temperature does not exceed −65° C. (30 min). A red suspension is formed. After addition is complete, the suspension is stirred at −75° C. for further 30 min. 1/3 of a solution of 145 g of methyl (2R,3S)-2,3-O-isopropylidene-3-phenylpropionate (ee: 99.05%, Daicel Chiralcel HPLC) in 150 ml of dry THF is then slowly added dropwise at a temperature below −65° C. during the course of 30 min. The residual quantity is then briskly added (5 min), a temperature rise to −60° C. taking place. After addition is complete the cooling bath is removed. On reaching an internal temperature of −30° C., 20 ml of methanol are added and at an internal temperature of 0° C. 200 ml of distilled water are added. The aqueous phase is separated off in a separating funnel, the organic phase is washed five times with 100 ml of distilled water each time, then the organic phase is extracted three times with 10% strength sulfuric acid (200 ml, 50 ml, 50 ml). The sulfuric acid phases are combined, treated with 200 ml of dichloromethane and adjusted to pH 2.3 with 10N sodium hydroxide solution and with ice cooling and vigorous stirring. The organic layer is separated off. The aqueous phase is extracted with 30 ml of dichloromethane. The combined dichloromethane phases are washed twice with a little distilled water. The organic layer is then dried over anhydrous sodium sulfate and the solvent is completely stripped off in vacuo. A brown oil is obtained which is treated with 50 ml of diethyl ether. After seeding, crystals are formed which are filtered off after standing overnight and washed with diethyl ether. After drying in vacuo, 57.7 g (52.5%, ee>99%, Daicel Chiralcel HPLC) of the title compound of melting point 76-80° C. are obtained as a pale yellow powder.

### A2. 2,3-Dimethyl-7-[(2S,3R)-2,3-O-isopropylidene-3-phenylpropan-1-on-1-yl]-8-pivaloylamino-imidazo[1,2-a]pyridine

The title compound (ee: 98.3%, Daicel Chiralcel HPLC) is obtained similarly to the procedure described in example A1 by using methyl (2S,3R)-2,3-O-isopropylidene-3-phenylpropionate (ee: 98%, Daicel Chiralcel HPLC) as acylating agent.

### B1. (8R,9R)-2,3-Dimethyl-8-hydroxy-9-phenyl-7,8,9,10-tetrahydroimidazo[1,2-h][1,7] naphthyridin-7-one

10.8 g (24 mmol) of 2,3-dimethyl-7-[(2R,3S)-2,3-O-isopropylidene-3-phenyipropan-1-on-1-yl]-8-pivaloylaminoimidazo[1,2-a]pyridine (ee>95%, Daicel Chiralcel HPLC) are introduced into 50 ml of 70% strength sulfuric acid with ice cooling during the course of 4 min. A suspension is formed in the course of this, which turns into an orange solution after 30 min. After addition is complete, the ice bath is removed and the mixture is stirred on at room temperature. The reaction solution is added after 50 h to ice water and dichloromethane is added, then the mixture is adjusted to pH 8 using 6N sodium hydroxide solution and saturated sodium hydrogen-carbonate solution. The organic phase is separated off. The aqueous phase is extracted twice with dichloromethane. The organic phases are combined and washed with a little distilled water. The organic layer is then dried over anhydrous sodium sulfate, filtered and concentrated on a vacuum rotary evaporator. The concentrated residue is chromatographed on silica gel (eluent: dichloromethane/methanol 100/1). The main fraction is concentrated and treated with ethyl acetate, and the title compound crystallizes in the course of this as a yellow solid. This precipitate is filtered off with suction and dried to constant weight in a vacuum drying oven at 50° C. 4.22 g (57%, ee>95%, Daicel Chiralcel HPLC) of the title compound of melting point 231-234° C. are obtained.

### B2. (8S,9S)-2,3-Dimethyl-8-hydroxy-9-phenyl-7,8,9,10-tetrahydroimidazo[1,2-h][1,7] naphthyridin-7-one

The title compound (ee: 94.0%, Daicel Chiralcel HPLC) is obtained according to the procedure described in example B1 starting from 2,3-dimethyl-7-[(2S,3R)-2,3-O-isopropylidene-3-phenylpropan-1-on-1-yl]-8-pivaloylaminoimidazo[1,2-a]pyridine.

### C1. (7R,8R,9R)-2,3-Dimethyl-7,8-dihydroxy-9-phenyl-7,8,9,10-tetrahydroimidazo[1,2-h][1,7]-naphthyridine

6 g (19.52 mmol) of (8R,9R)-2,3-dimethyl-8-hydroxy-9-phenyl-7,8,9,10-tetra-hydroimidazo-[1,2-h][1,7]naphthyridin-7-one (ee>90%, Daicel Chiralcel HPLC) are suspended in 60 ml of methanol and cooled to −5 to 0° C. in a methanol-ice bath. At this temperature, sodium borohydride (0.81 g, 21.47 mmol) is added by spatula during the course of 0.5 h (evolution of gas). After addition is complete, the mixture is stirred for a further 10 min, and then concentrated in a vacuum rotary evaporator at a bath temperature of 40° C. The oily residue obtained is taken up in distilled water and extracted three times with chloroform. The organic phases are combined and washed with a little water, then dried using anhydrous sodium sulfate and filtered. The filtrate is concentrated on a vacuum rotary evaporator and co-evaporated with acetone; the title compound crystallizes out in the course of this. The precipitate is filtered off, washed with acetone and dried to constant weight at 50° C. in a vacuum drying oven. 5.15 g (85.3%, ee>90%, Daicel Chiralcel HPLC) of the title compound are obtained as a colorless crystallizate of melting point 206-9° C.

### C2. (7S,8S,9S)-2,3-Dimethyl-7,8-dihydroxy-9-phenyl-7s8,9f 1o-tetrahydroimidazo [1,2-h][1,7] naphthyridine

The title compound of mp 207-208° C. (ee: 98.7%, Daicel Chiralcel HPLC) is obtained according to the procedure described in example C1 using (8S,9S)-2,3-dimethyl-8-hydroxy-9-phenyl-7,8,9,10-tetrahydroimidazo [1,2-h][1,7] naphthyridin-7-one as starting material.

### D. (7S,8R,9R)-2,3-Dimethyl-7,8-dihydroxy-9-phenyl-7,8,9,10-tetrahydro-imidizo[1,2-h][1,7]naphthyridine

2 g of the mother liquor of Example C1 are chromatographed on silica gel (eluent:ethyl acetate/methanol 19/1) to give 0.35 g of the title compound as an oil which crystallizes upon addition of ethyl acetate. Melting point: 199-200° C. (ethyl acetate).

The pharmaceutical composition according to the invention is prepared by processes known per se, which are familiar to the person skilled in the art. In the pharmaceutical composition, the pharmacologically active compounds according to the invention are employed either as such, or preferably in combination with suitable pharmaceutical auxiliaries or excipients in the form of intraocular devices, where the active compound content is advantageously and where, by the appropriate choice of the auxiliaries and excipients, a pharmaceutical administration form exactly suited to the active compound and/or to the desired onset of action can be achieved.

The person skilled in the art is familiar, on the basis of his expert knowledge, with auxiliaries or excipients which are suitable for the desired pharmaceutical formulations.

The active compounds are preferably administered orally, topically, intravitreally, subretinally or periocularly. It has proven advantageous to administer the active compound (s) in a dose from 10-50 ng/ml. Favourably a dosage of about 10 to about 50 mg/kg body weight, in particular about 10 to about 40 mg/kg, more preferably of about 10 to about 36 mg/kg body weight is administered to the patient. The optimal dose and manner of administration of the active compounds necessary in each case can easily be determined by any person skilled in the art on the basis of his expert knowledge.

If the compounds according to the invention and/or their salts are to be employed for the treatment of the above mentioned diseases, the pharmaceutical preparations can also contain one or more pharmacologically active constituents of other pharmaceutical groups.

Soraprazan was administrated in oral application of 6, 12 and 24 mg/kg/day for 52 weeks in the Cynomolgus monkey.

Some monkeys from the control and the high-dose group were subjected to a recovery period of 3 months.

Conventional histopathology revealed no alterations after treatment with 6 and 12 mg/kg/day. Three out of 12 monkeys treated with 24 mg/kg/day, including 1 animal with a funduscopic abnormality, showed migration of individual macrophages either beneath the RPE (1 animal), and/or into the subretinal space (3 animals). Two of these 3 monkeys had depigmentation of RPE cells although the photoreceptors facing the depigmented RPE stayed healthy. These RPE cells had released melanin as well as lipofuscin granules to secondary cells that had migrated between Bruch's membrane and the RPE cell layer or into the subretinal space.

Therefore this shows that it is possible to stop the progression of lipofuscin accumulation in conditions where there is a risk of getting Stargardt's disease.

As lipofuscin can be easily detected in the fundus, the invention would also allow prevention of the disease, as detection can already be done at an early stage of the disease development.

### Description of Examples

### Removal of Lipofuscin from RPE Cells in ABCR Knock-Out Mice after Treatment with Soraprazan

### Study in ABCR Knock-Out Mice: Overview

| Type of Study | Pharmacodynamic study in vivo |
|---|---|
| Objectives | 1. To investigate degradation of lipofuscin with Soraprazan in vivo in a |
| | Stargardt's disease model after local administration. |
| | 2. To investigate ultra-structural changes induced by treatment with |
| | Soraprazan. |
| Methods | ABCA4^{-/-} mice, 2 groups of 3 (treated) and 2 (control) animals each |
| | Single injection of 2 μl PBS containing 0.32 mg Soraprazan in |
| | suspension |
| | Quantitative analysis of 44 different retinal areas for RPE lipofuscin |
| | content. |
| | Ultra-structural analysis of TEM sections |
| | Qualitative analysis of lipofuscin content with fluorescence microscope |
| Results | 1. Lipofuscin is removed from ABCA4^{-/-} mouse RPE cells after |
| | treatment with Soraprazan. The effect is statistically significant at the |
| | p < 0.0001 level. |
| | 2. Macrophages filled with pigmented particles were occasionally |
| | observed in the choroid and adjacent to RPE cells and Bruch's |
| | membrane. |
| Conclusions | The treatment of ABCA4^{-/-} mice with Soraprazan leads to the removal |
| | of lipofuscin from RPE cells. Macrophages filled with pigment particles |
| | including lipofuscin may be involved in the process of degrading and |
| | removing lipofuscin. Retinas of treated animals made a healthy |
| | appearance. Soraprazan treatment and the subsequent removal of |
| | lipofuscin form RPE cells may prove to be beneficial for patients with |
| | Stargardt's disease. |

### Objective

ABCA4 knock-out mice were treated with an intravitreal injection of Soraprazan.

The study focus was set on lipofuscin content in RPE cells, ultra-structure of the outer retina and auto-fluorescence properties of RPE cells. The ABCA4^{-/-} model for Stargardt's disease was recently characterized by Charbel Issa and co-workers (Charbel Issa et al. 2013).

### Methods

Study Design for Intravitreal Injection of Soraprazan into ABCR^{-/-} Mice:

Pigmented ABCA4^{-/-} mice (129S4/SvJae-ABCA4^{tm1Ght}) were provided by Gabriel Travis (David Geffen School of Medicine, University of California, Los Angeles, Calif.) and bred in the Biomedical Sciences division, University of Oxford. All experiments were conducted in female mice. The animals were kept in a 12 hour light (<100 lux)/dark cycle, with food and water available ad libitum. All animal breeding and experimental procedures were performed under approval of local and national ethical and legal authorities, and were conducted in compliance with the ARVO statement for the Use of Animals in Ophthalmic and Vision Research.

### Surgery

Two μl PBS containing a total amount of 0.32 mg Soraprazan in suspension were injected into the vitreous of three 12 months old ABCA4^{-/-} mice.

### Preparation and Embedding for Kight and Electron Microscopy

The eyes were enucleated after four weeks. Two untreated aged matched ABCA4^{-/-} mice served as controls. After enucleation, the eyes were cleaned of orbital tissue and, after removal of the cornea, were fixed overnight at 4° C. in 2% glutaraldehyde in 0.1 M cacodylate buffer (pH 7.4) containing 100 mM sucrose. After washing with cacodylate buffer, areas of interest in flat mount preparations were excised and post-fixed with 1% osmium tetroxide in 0.1 M cacodylate buffer at room temperature for 1 h. In some parts of the eyes osmication was omitted which preserved the autofluorescence of lipofuscin. Dehydration was then started by a series of incubations in 30, 50, and 70% ethanol. The samples were stained with saturated uranyl acetate. Dehydration was continued by incubations in 70, 80, 95, 100% ethanol and propylene oxide. The samples were then embedded in Epon (SPI-Pon™812 Epoxy Embedding Kit, SPI supplies, West Chester, Pa.). For light and fluorescence microscopy, staining with osmium and uranylacetate was omitted.

### Electron Microscopy and Pigment Granule Quantification

Ultrathin Epon sections (70 nm) from WT control eyes and ABCA4^{-/-} eyes were post-stained with lead citrate and investigated under a transmission electron microscope (TEM; Model 902 A, Carl Zeiss, Oberkochen, Germany). For statistical analysis, the areas occupied by lipofuscin granules were quantified in 8-10 micrographs from each group. As lipofuscin is somewhat unusual in ABCA4^{-/-} mice, the morphological definition we used in this study of the RPE was as follows: lipofuscin in ABCA4^{-/-} mice is a type of intracellular granule which appears in the electron microscope as a membrane bound body with heterogeneous staining and very variable shape generally darker than the cytosol. Lipofuscin in the ABCA4 mice often fuses with melanin granules. Melanin was excluded from the measurements. For lipofuscin quantification, image analysis software (iTEM, Olympus Soft imaging Solutions, Munster, Germany) was used. For each image, the total area of RPE cytoplasm was determined. Nuclei were not included in this measurement. Apical microvilli and extracellular space in the region of the basal infoldings were also excluded. The included area totalled from about 50-100 μm² per image. The area fraction of lipofuscin/melanolipofuscin is expressed as area in μm² occupied by lipofuscin per 100 μm² RPE cytoplasm.

### Fluorescence Microscopy

Autofluorescent lipofuscin and melanolipofuscin granules were photographed in semi thin sections using a fluorescence microscope (Zeiss Axioplan2 imaging, excitation 370/36 nm, emission 575/15 nm, 40× objective) connected to a personal computer equipped with a CDD camera. Since the granules could not clearly be separated from each other and lipofuscin cannot be differentiated from melanolipofuscin, the quantification of lipofuscin granules was performed using electron microscopy.

### Statistical Analysis

Statistical significance for the ultrastructural evaluation of lipofuscin granules was determined by using Dunnett's test and the JMP9 statistical program (SAS; Heidelberg, Germany). A p-value of <0.05 was considered statistically significant.

### Results

### Fluorescence Microscopy

Semithin sections qualitatively illustrate the different degree of lipofuscin pigmentation in RPE of ABCA4^{-/-} with (FIG. 2 A) and without treatment (FIG. 2 B). In the RPE of 12-month-old ABCA4^{-/-} mice, bright dots (arrows) representing lipofuscin were detected. Under fluorescent light lipofuscin was reduced after treatment with Soraprazan (FIG. 2 A) compared to RPE from untreated mice (FIG. 2 B).

FIG. 2 shows under fluorescent light lipofuscin granules (arrows) in RPE of ABCA4−/− mice, which are reduced in number 4 weeks after a single intravitreal injection of 0.32 mg Soraprazan (A) compared to untreated mice (B).

### Ultrastructural Observations in the TEM

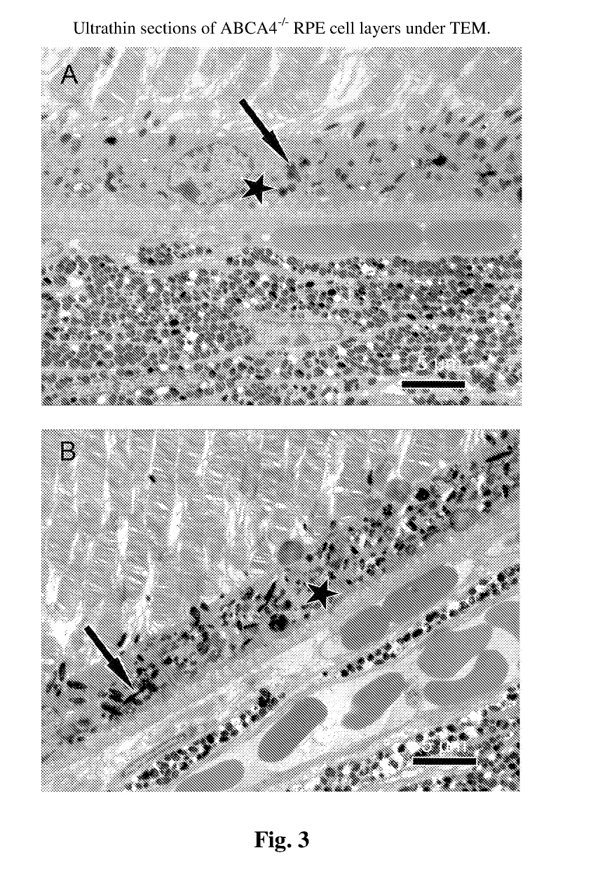

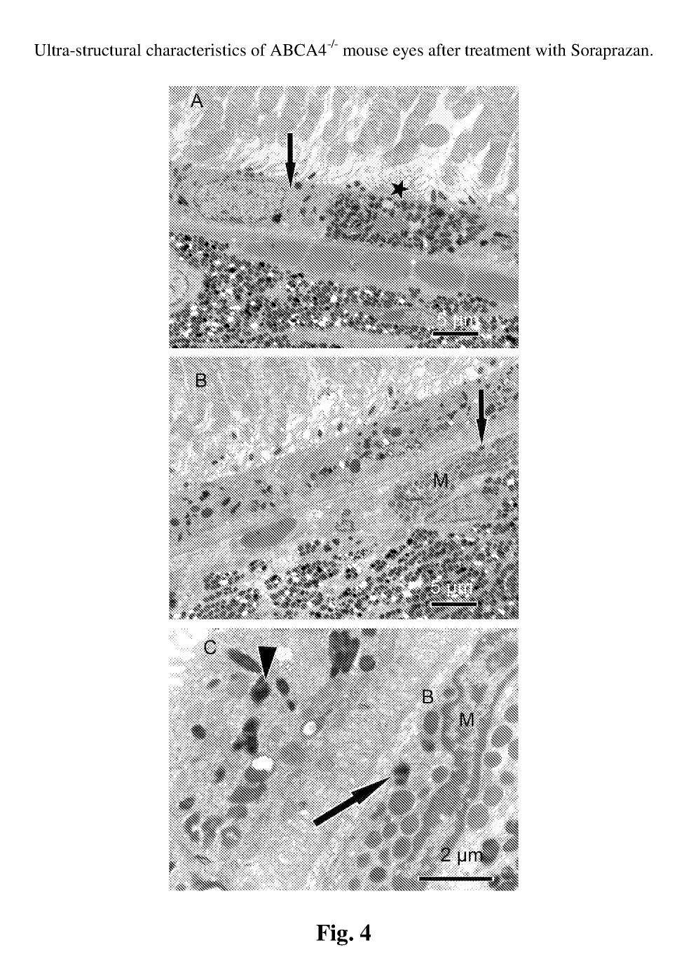

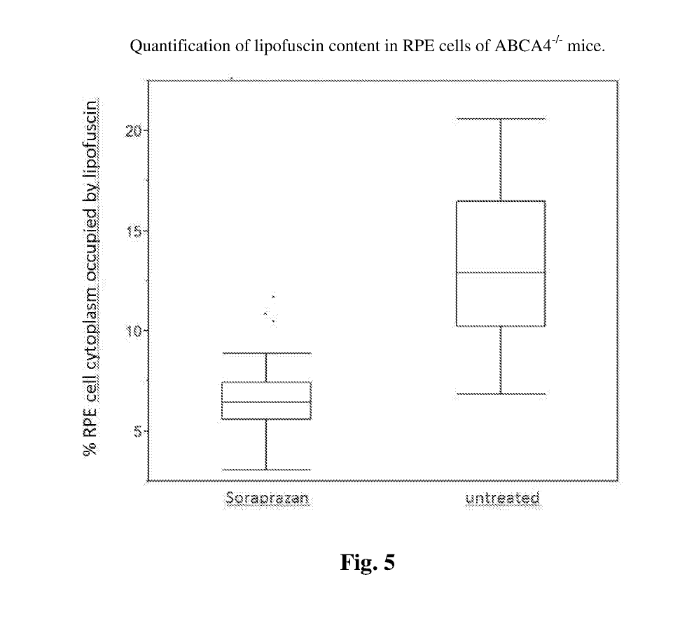

The retina, RPE and choroid tissues were examined to determine ultra-structural differences in Soraprazan-treated and non-treated (control) ABCA4^{-/-} mice. Prominent differences were only detected in the RPE. In Soraprazan treated ABCA4^{-/-} mice, electron-opaque lipofuscin granules as shown in FIG. 3 A were significantly reduced in number compared to controls (FIG. 3 B and FIG. 5). In treated mice occasionally individual RPE cells were observed that did not lost significant amounts of lipofuscin (FIG. 4 A). This however, very impressively showed how huge the amount of lipofuscin was, that could be removed in the majority of the RPE cells (FIG. 4 A). In untreated mice the amount of lipofuscin was always homogenous in all RPE cells. In treated mice macrophages were observed that were located directly below Bruch's membrane and contained lipofuscin granules (FIGS. 4 B and C). This suggests that lipofuscin was transported away by active macrophages after phagocytosis.

FIG. 3 shows TEM micrographs of RPE cells from ABCA4^{-/-} mice with Soraprazan treatment (FIG. 3 A) and untreated (FIG. 3 B). With progression of age unusual lipofuscin granules of irregular shape and electron density (black arrows) accumulate in the RPE (asterisk) cytoplasm of 12-month-old ABCA4^{-/-} mice.

In FIG. 4 TEM micrographs of the RPE from 12-month-old ABCA4^{-/-} mice are shown after a single intravitreal injection of Soraprazan. Different degree of lipfuscin removal is shown

FIG. 4 A shows the RPE cell on the left marked by an arrow, which has lost most of its lipofuscin whereas the adjacent RPE cell on the right (asterisks) still contains high amounts of lipofuscin. FIG. 4 B shows a macrophage (M), which is localized directly below Bruch's membrane (B) and contains high amounts of partly degraded pigment granules (arrow). This macrophage is shown at higher magnification in FIG. 4 C and the arrow marks the same lipofuscin granule as in FIG. 4 B. Typical lipofuscin granules are present within the macrophage (arrow) and within the RPE cell cytoplasm (arrowhead).

### Quantification of Lipofuscin Granules by Electron Microscopy

The percent area occupied by lipofuscin in RPE cell cytoplasm was higher in untreated ABCA4^{-/-} mice (median 12.92%) compared to age-matched Soraprazan treated mice (median 6.42%; FIG. 5). This reduction was statistically significant (p<0.0001, Dunnett's test).

FIG. 5 shows a Quantification of lipofuscin granules by electron microscopy in RPE cells of ABCA4^{-/-} mice. 44 different retinal areas from five different animals were analyzed. The percent area occupied by lipofuscin in sectioned RPE cytoplasm significantly decreased in Soraprazan treated ABCA4^{-/-} mice vs. untreated controls (p<0.0001, Dunnett's test).

These results demonstrate that administration of Soraprazan induces lipofuscin elimination from RPE cells. Observations in a Stargardt's disease murine model include:
the qualitative removal of lipofuscin after treatment with Soraprazan observed in fluorescence images,
the highly significant quantitative reduction of lipofuscin content in ultrathin TEM sections after treatment with Soraprazan, and
macrophages transporting lipofuscin granules in the choroid of Soraprazan treated animals and thereby partly explaining a mechanism of degradation and removal.

### LITERATURE

Allikmets, R, N F Shroyer, N Singh, J M Seddon, R A Lewis, P S Bernstein, A Peiffer, et al. 1997. “Mutation of the Stargardt Disease Gene (ABCR) in Age-related Macular Degeneration.” Science (New York, N.Y.) 277 (5333) (September 19): 1805-1807.
Blacharski, P. A. 1988. “Fundus Flavimaculatus.” In Retinal Dystrophies and Degenerations, 135-159. New York: Raven Press.
Burke, Tomas R, and Stephen H Tsang. 2011. “Allelic and Phenotypic Heterogeneity in ABCA4 Mutations.” Ophthalmic Genetics 32 (3) (September): 165-174. doi:10.3109/13816810.2011.565397.
Charbel Issa, Peter, Alun R Barnard, Mandeep S Singh, Emma Carter, Zhichun Jiang, Roxana A Radu, Ulrich Schraermeyer, and Robert E Maclaren. 2013. “Fundus Autofluorescence in the Abca4−/− Mouse Model of Stargardt Disease—Correlation with Accumulation of A2E, Retinal Function and Histology.” Investigative Ophthalmology & Visual Science (June 11). doi:10.1167/iovs.13-11688.
Chen, Yingming, Kavitha Ratnam, Sanna M. Sundquist, Brandon Lujan, Radha Ayyagari, V. Harini Gudiseva, Austin Roorda, and Jacque L. Duncan. 2011. “Cone Photoreceptor Abnormalities Correlate with Vision Loss in Patients with Stargardt Disease.” Investigative Ophthalmology & Visual Science 52 (6) (January 5): 3281-3292. doi:10.1167/iovs.10-6538.
Genead, Mohamed A., Gerald A. Fishman, Edwin M. Stone, and Rando Allikmets. 2009. “The Natural History of Stargardt Disease with Specific Sequence Mutation in the ABCA4 Gene.” Investigative Ophthalmology & Visual Science 50 (12) (January 12): 5867-5871. doi:10.1167/iovs.09-3611.
Hamel, Christian P. 2007. “Cone Rod Dystrophies.” Orphanet Journal of Rare Diseases 2 (February 1): 7. doi:10.1186/1750-1172-2-7.
Julien, Sylvie, and Ulrich Schraermeyer. 2012. “Lipofuscin Can Be Eliminated from the Retinal Pigment Epithelium of Monkeys.” Neurobiology of Aging 33 (10) (January 12): 2390-2397. doi:10.1016/j.neurobiolaging.2011.12.009.
Kennedy, C J, P E Rakoczy, and I J Constable. 1995. “Lipofuscin of the Retinal Pigment Epithelium: a Review.” Eye (London, England) 9 (Pt 6): 763-771.
Ng, Kwok-Peng, Bogdan Gugiu, Kutralanathan Renganathan, Matthew W Davies, Xiaorong Gu, John S Crabb, So Ra Kim, et al. 2008. “Retinal Pigment Epithelium Lipofuscin Proteomics.” Molecular & Cellular Proteomics: MCP 7 (7) (July): 1397-1405. doi:10.1074/mcp.M700525-MCP200.
Openshaw, Amanda, Kari Branham, and John Heckenlively. 2008. “Understanding Stargardt Disease”. University of Michigan Kellog Eye Center. http://www.kellogg.umich.edu/patientcare/downloads/Understand-Stargardt.pdf.
Ottis, Philipp, Katharina Koppe, Bruce Onisko, Irina Dynin, Thomas Arzberger, Hans Kretzschmar, Jesus R Requena, Christopher J Silva, Joseph P Huston, and Carsten Korth. 2012. “Human and Rat Brain Lipofuscin Proteome.” Proteomics 12 (15-16) (August): 2445-2454. doi:10.1002/pmic.201100668.
Terman, A, and U T Brunk. 1998. “Lipofuscin: Mechanisms of Formation and Increase with Age.” APMIS: Acta Pathologica, Microbiologica, et Immunologica Scandinavica 106 (2) (February): 265-276.
Vasireddy, Vidyullatha, Paul Wong, and Radha Ayyagaria. 2010. “Genetics and Molecular Pathology of Stargardt-like Macular Degeneration.” Progress in Retinal and Eye Research 29 (3) (May): 191-207. doi:10.1016/j.preteyeres.2010.01.001.

1. (canceled)

2. (canceled)

3. A method of treatment of Stargardt's disease in a subject, the method comprising administering to the subject in need of such treatment a pharmaceutical composition comprising a compound according to the following formula I 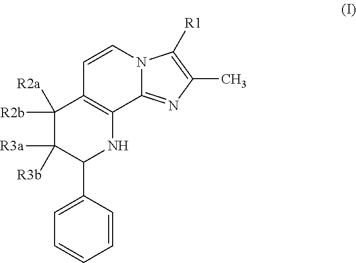
in which R1 is methyl or hydroxymethyl, one of the substituents R2a and R2b is hydrogen and the other is hydroxy, methoxy, ethoxy, isopropoxy, methoxyethoxy or ethoxypropoxy, one of the substituents R3a and R3b is hydrogen and the other is hydroxy, methoxy, ethoxy, isopropoxy, methoxyethoxy or methoxypropoxy, where R2a or R2b on the one hand and R3a or R3b on the other hand are not simultaneously hydroxy, and its salts.

4. The method of claim 3, in which R1 is methyl, one of the substituents R2a and R2b is hydrogen and the other is methoxy, ethoxy, isopropoxy, methoxyethoxy or methoxypropoxy, one of the substituents R3a and R3b is hydrogen and the other is hydroxy, and its salts.

5. The method of claim 3, in which R1 is methyl, one of the substituents R2a and R2b is hydrogen and the other is methoxy, ethoxy, isopropoxy, methoxyethoxy or methoxypropoxy, one of the substituents R3a and R3b is hydrogen and the other is methoxy, ethoxy, isopropoxy, methoxyethoxy or methoxypropoxy, and its salts.

6. The method of claim 3, in which R3b is hydrogen.

7. The method of claim 3, in which R2a and R3b are hydrogen.

8. The method of claim 3, in which R2a is hydrogen and R2b is methoxy, ethoxy, isopropoxy, methoxyethoxy or methoxypropoxy, and its salts.

9. A method of treatment of Stargardt's disease in a subject, comprising administering to the subject in need of such treatment a pharmaceutical composition comprising a compound according to the following formula I* 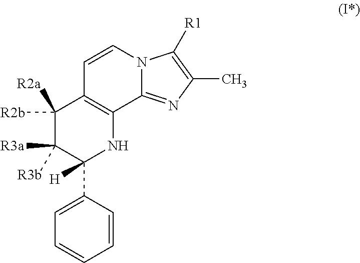
in which R1 is methyl or hydroxymethyl, one of the substituents R2a and R2b is hydrogen and the other is hydroxy, methoxy, ethoxy, isopropoxy, methoxyethoxy or ethoxypropoxy, one of the substituents R3a and R3b is hydrogen and the other is hydroxy, methoxy, ethoxy, isopropoxy, methoxyethoxy or methoxypropoxy, where R2a or R2b on the one hand and R3a or R3b on the other hand are not simultaneously hydroxy, and its salts.

10. The method of claim 9, in which R1 is methyl, one of the substituents R2a and R2b is hydrogen and the other is methoxy, ethoxy, isopropoxy, methoxyethoxy or methoxypropoxy, one of the substituents R3a and R3b is hydrogen and the other is hydroxy, and its salts.

11. The method of claim 9, in which R1 is methyl, one of the substituents R2a and R2b is hydrogen and the other is methoxy, ethoxy, isopropoxy, methoxyethoxy or methoxypropoxy, one of the substituents R3a and R3b is hydrogen and the other is methoxy, ethoxy, isopropoxy, methoxyethoxy or methoxypropoxy, and its salts.

12. The method of claim 9, in which R3b is hydrogen.

13. The method of claim 9, in which R2a and R3b are hydrogen.

14. A method of treatment of Stargardt's disease in a subject, the method comprising administering to the subject in need of such treatment a pharmaceutical composition comprising a compound according to the following formula I** 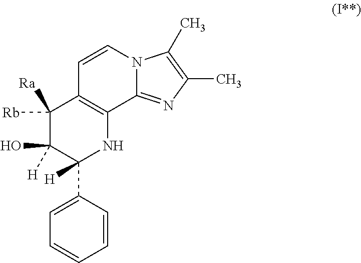
in which one of the substituents Ra and Rb is hydrogen and the other is methoxy, ethoxy, isopropoxy, methoxyethoxy or methoxypropoxy, and its salts.

15. The method of claim 14, in which Ra is hydrogen and Rb is methoxy, ethoxy, isopropoxy, methoxyethoxy or methoxypropoxy, and its salts.

16. The method of claim 3, wherein the compound according to the following formula I is (7R,8R,9R)-2,3-Dimethyl-8-hydroxy-7-(2-methoxyethoxy)-9-phenyl-7,8,9,10-tetrahydro-imidazo[1,2-h][1,7]naphthyridine and its salts or (7R,8R,9R)-2,3-Dimethyl-7-ethoxy-8-hydroxy-9-phenyl-7,8,9,10-tetrahydro-imidazo [1,2-h][1,7] naphthyridine and its pharmacologically acceptable salts.

17. The method of claim 9, wherein the compound according to the following formula I* is (7R,8R,9R)-2,3-Dimethyl-8-hydroxy-7-(2-methoxyethoxy)-9-phenyl-7,8,9,10-tetrahydro-imidazo[1,2-h][1,7]naphthyridine and its salts or (7R,8R,9R)-2,3-Dimethyl-7-ethoxy-8-hydroxy-9-phenyl-7,8,9,10-tetrahydro-imidazo[1,2-h][1,7] naphthyridine and its pharmacologically acceptable salts.

18. The method of claim 14, wherein the compound according to the following formula I** is (7R,8R,9R)-2,3-Dimethyl-8-hydroxy-7-(2-methoxyethoxy)-9-phenyl-7,8,9,10-tetrahydro-imidazo[1,2-h][1,7]naphthyridine and its salts or (7R,8R,9R)-2,3-Dimethyl-7-ethoxy-8-hydroxy-9-phenyl-7,8,9,10-tetrahydro-imidazo[1,2-h][1,7] naphthyridine and its pharmacologically acceptable salts.
